(19) United States
(12) Patent Application Publication
(10) Pub. No.: US 2009/0270733 A1
(43) Pub. Date: Oct. 29, 2009

(54) ULTRASONIC IMAGING APPARATUS AND METHOD

(76) Inventor: Tetsuo Koide, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/429,402

(22) Filed: Apr. 24, 2009

(30) Foreign Application Priority Data
Apr. 25, 2008 (JP) 2008-115243

(51) Int. Cl. A61B 8/14

(52) U.S. Cl. 600/447; 600/458

(57) ABSTRACT

An ultrasonic imaging method using an ultrasonic probe for applying a first ultrasonic beam to a first region of three-dimensional region of a subject with a contrast agent administered thereto and acquiring three-dimensional tomographic image information for the first region, including the steps of: setting a second region for applying a second ultrasonic beam whose sound pressure is higher than that of the first ultrasonic beam for not destroying the contrast agent, in the three-dimensional region; and applying the second ultrasonic beam to exceed sound pressure for destroying the contrast agent only in the second region and performing the irradiation of the second ultrasonic beam by the ultrasonic probe in the course of acquisition of the three-dimensional tomographic image information in the first region by the first ultrasonic beam.

### CROSS REFERENCE TO RELATED APPLICATIONS

This application claims the benefit of Japanese Patent Application No. 2008-115243 filed Apr. 25, 2008, which is hereby incorporated by reference in its entirety.

### BACKGROUND OF THE INVENTION

The present invention relates to an ultrasonic imaging apparatus for imaging a contrast agent administered to a subject using a three-dimensional ultrasonic probe.

With the recent advancement of image diagnosis equipment, morphologic tomographic image information in a subject can be acquired at a high resolution. An operator performs various measurements of targeted portions or regions of the subject, using these morphologic tomographic image information and acquires useful diagnostic information (see, for example, “Revised Medical Ultrasonic Equipment Handbook” edited by Electronic Industries Association of Japan, issued by Corona Publishing Co., Ltd., Jan. 20 1997, p. 134-139).

Further, the operator is also able to estimate the state of morphologically indistinguishable regions or regions by simulation on the basis of these measured information. When, for example, the liver is removed due to a disease such as a tumor, it is necessary to determine a range to be removed. Here, the range to be removed is decided based on information on dominant regions or regions of vascular channels determined by simulation from the morphologic positions of the vascular channels that branch off from an arterial vessel run within the liver.

FIG. 18 is an explanatory diagram typically showing vascular channels 2 through 4 run inside a liver 1. The vascular channels 2 through 4 are branched ones of an arterial vessel that enters from a portal vein to the liver. Blood flows that entered into the vascular channels 2 through 4 are absorbed from the peripheral blood vessels of the vascular channels 2 through 4 to liver's tissues. Thereafter, the blood flows are eliminated from venous vessels having structures similar to the vascular channels 2 through 4 to the outside of the liver.

Here, the blood flows absorbed from the peripheral blood vessels of the vascular channels 2 through 4 to the liver's tissues are mainly used in dominant regions or regions that exist in the neighborhood of the peripheral blood vessels of the vascular channels 2 through 4. When the liver 1 is partly removed, disease-free dominant regions preferably remain without injury for the post-removal liver. It is thus important to recognize the respective dominant regions set every vascular channel constituting the liver when the liver 1 is partly removed.

Upon the above simulation, the dominant regions are determined by calculation from such a tomographic image of liver 1 as shown in FIG. 18, which is photographed or imaged using an X-ray CT apparatus or the like. In this calculation, for example, the positions of the vascular channels 2 and 3 in the direction orthogonal to the flow of blood are determined, and the midpoint therebetween is defined as a boundary between the dominant regions that the vascular channels 2 and 3 have. In FIG. 18, the dominant regions 5 through 7 of the vascular channels 2 through 4, which have been determined by calculation in this way, are illustrated as regions or regions surrounded by broken lines.

According to the background art, however, there is a case in which the dominant regions of the vascular channels differ from actual ones. That is, the dominant regions are equivalent to ones determined by calculation and are of merely estimated ones strictly. In the neighborhood of the boundary between the dominant regions in particular, errors were contained in the dominant regions.

Information about each dominant region becomes a turning point whether a healthy dominant region remains without injury where the liver is partly removed. The information exerts an influence on whether the prognosis of the subject becomes satisfactory.

### BRIEF DESCRIPTION OF THE INVENTION

An ultrasonic imaging apparatus according to a first aspect includes an ultrasonic probe for applying a first ultrasonic beam to a first region of three-dimensional region of a subject with a contrast agent administered thereto and acquiring three-dimensional tomographic image information for the first region, including: a region setting device for setting a second region for applying a second ultrasonic beam whose sound pressure is higher than that of the first ultrasonic beam for not destroying the contrast agent, in the three-dimensional region; and a controller for allowing the second ultrasonic beam to exceed sound pressure for destroying the contrast agent only in the second region and performing the irradiation of the second ultrasonic beam by the ultrasonic probe in the course of acquisition of the three-dimensional tomographic image information in the first region by the first ultrasonic beam.

In the first aspect, a second region for applying a second ultrasonic beam of high sound pressure is set to three-dimensional tomographic image information. The irradiation of the second ultrasonic beam set so as to exceed sound pressure for destroying a contrast agent only in the second region is performed in the course of acquisition of three-dimensional tomographic image information using a first ultrasonic beam.

A second aspect is provided wherein in the ultrasonic imaging apparatus described in the first aspect, the second region is part of blood vessels that branch off within the liver of the subject.

In the second aspect, a contrast agent that flows into vascular channels is destroyed by a second ultrasonic beam.

A third aspect is provided wherein in the ultrasonic imaging apparatus described in the first or second aspect, the region setting device sets the second region to two-dimensional tomographic image information constituting the three-dimensional tomographic image information, and the ultrasonic imaging apparatus also includes a display unit for displaying an image based on the two-dimensional tomographic image information, and an irradiation section setting device for setting an irradiation sectional region for the second region to the image.

In the third aspect, an irradiation sectional region shown in two-dimensional tomographic image information for a second region is set.

A fourth aspect is provided wherein in the ultrasonic imaging apparatus described in the third aspect, the irradiation section setting device sets marker regions for detecting the motion of the irradiation sectional region to the image.

In the fourth aspect, marker regions are respectively set to locations where it is easy to detect the motion of an irradiation sectional region.

A fifth aspect is provided wherein in the ultrasonic imaging apparatus described in the fourth aspect, the region setting device has a marker region position detection device for detecting a location where each of the marker regions is positioned.

In the fifth aspect, the detection of motion of an irradiation sectional region is ensured.

A sixth aspect is provided wherein in the ultrasonic imaging apparatus described in the fifth aspect, the region setting device has an irradiation section resetting device for resetting the position of the irradiation sectional region, based on information about the positions of the marker regions detected by the marker region position detection device.

In the sixth aspect, an irradiation sectional region is moved according to the motion of a subject.

A seventh aspect is provided wherein in the ultrasonic imaging apparatus described in any one of the third through sixth aspects, the region setting device has an irradiation region generation device for expanding the irradiation sectional region in a thickness direction orthogonal to the image and thereby generating the second region.

In the seventh aspect, an irradiation sectional region is expanded in a thickness direction and thereby a second region is generated.

An eighth aspect is provided wherein in the ultrasonic imaging apparatus described in the seventh aspect, the irradiation region generation device has a region limit device for limiting the length of the second region in the thickness direction to within a predetermined restricted distance.

In the eighth aspect, the length of a second region in a thickness direction is fit within a predetermined range.

A ninth aspect is provided wherein the ultrasonic imaging apparatus described in the eighth aspect also includes a restricted distance input key for inputting the restricted distance.

In the ninth aspect, the length of a second region in a thickness direction can be inputted.

A tenth aspect is provided wherein in the ultrasonic imaging apparatus described in any one of the third through ninth aspects, the controller has an irradiation sectional region measuring device for measuring the size of the irradiation sectional region in an electronic scan direction orthogonal to the direction of a depth that the image based on the two-dimensional tomographic image information has.

In the tenth aspect, the size of a region for irradiating a second ultrasonic beam is determined.

An eleventh aspect is provided wherein in the ultrasonic imaging apparatus described in any one of the third through tenth aspects, the controller has a sound pressure distribution calculation device for calculating a sound pressure distribution of a second ultrasonic beam generated by a sound ray closest to a center position of the irradiation sectional region.

A twelfth aspect is provided wherein in the ultrasonic imaging apparatus described in the eleventh aspect, the sound pressure distribution calculation device sets a depth that the center position of the irradiation sectional region has, to a focal depth on which the second ultrasonic beam is focused.

In the twelfth aspect, the sound pressure distribution calculation device sets sound pressure to the maximum in an irradiation sectional region.

A thirteenth aspect is provided wherein in the ultrasonic imaging apparatus described in any one of the third through twelfth aspects, the controller has a sound pressure decision parameter determination device for changing sound pressure decision parameters used upon the calculation of the sound pressure distribution.

In the thirteenth aspect, sound pressure decision parameters are changed to determine an optimum sound pressure distribution of a second ultrasonic beam.

A fourteenth aspect is provided wherein in the ultrasonic imaging apparatus described in the thirteenth aspect, the sound pressure decision parameters include an aperture width and a drive voltage used when the second ultrasonic beam is transmitted.

In the fourteenth aspect, a sound pressure distribution is controlled by adjustments to an aperture width and a drive voltage.

A fifteenth aspect is provided wherein in the ultrasonic imaging apparatus described in the fourteenth aspect, the aperture width is set to an aperture width wider than the aperture width at the time that the first transmission is done.

In the fifteenth aspect, a sound pressure distribution is enhanced steeply in an irradiation sectional region located in a focal depth.

A sixteenth aspect is provided wherein in the ultrasonic imaging apparatus described in any one of the third through fifteenth aspects, the controller has a beam change device for stopping the first ultrasonic beam which acquires the two-dimensional tomographic image information at a position of the sound ray closest to the center position of the irradiation sectional region and generating the second ultrasonic beam in place of the first ultrasonic beam, upon acquisition of the three-dimensional tomographic image information.

In the sixteenth aspect, a contrast agent for a second region is destroyed while the acquisition of three-dimensional tomographic image information is being performed.

A seventeenth aspect is provided wherein in the ultrasonic imaging apparatus described in any one of the first through sixteenth aspects, the ultrasonic probe has a piezoelectric transducer array in which piezoelectric transducers are arranged on a one-dimensional basis, and a mechanical scan unit for mechanically moving the piezoelectric transducer array in the direction approximately orthogonal to the direction of the arrangement thereof.

In the seventeenth aspect, a one-dimensional piezoelectric transducer array is mechanically driven to acquire three-dimensional tomographic image information.

An eighteenth aspect is provided wherein in the ultrasonic imaging apparatus described in the seventeenth aspect, the controller stops the mechanical scan and repeatedly performs only an electronic scan done in the direction of the arrangement of the piezoelectric transducer array upon acquisition of the two-dimensional tomographic image information by the electronic scan.

In the eighteenth aspect, two-dimensional tomographic image information is acquired at a high frame rate while the destruction of a contrast agent is being performed.

A nineteenth aspect is provided wherein in the ultrasonic imaging apparatus described in any one of the first through eighteenth aspects, the ultrasonic probe has a two-dimensional piezoelectric transducer array in which piezoelectric transducers are two-dimensionally arranged at a surface brought into contact with the subject.

In the nineteenth aspect, three-dimensional tomographic image information is acquired by an electronic scan alone.

According to the invention, dominant regions or regions of vascular channels that constitute a liver are projected or drawn as low-brightness regions free of existence of a contrast agent. The dominant regions can be visually confirmed. By extension, when the subject's liver is partly removed, for example, it can be performed without injury of healthy dominant regions and the prognosis of the subject can be made satisfactory.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an overall construction of an ultrasonic imaging apparatus.

FIG. 2 is an external diagram illustrating a construction of an ultrasonic probe.

FIG. 3 is an explanatory diagram showing an electronic scan and a mechanical scan of the ultrasonic probe.

FIG. 4 is an explanatory diagram illustrating a three-dimensional imaging region acquired by the ultrasonic probe and its display sections.

FIG. 5 is a block diagram depicting a construction of a controller according to an embodiment.

FIG. 6 is a flowchart showing the operation of the controller according to the embodiment.

FIG. 7 is an explanatory diagram illustrating a branch portion of a hepatic artery displayed in an A section.

FIG. 8 is a flowchart depicting the operation of a high acoustic pressure irradiation region setting process according to the embodiment.

FIG. 9 is an explanatory diagram showing an irradiation sectional region set by an operator and a three-dimensional high sound or acoustic pressure irradiation region generated from the irradiation sectional region.

FIG. 10 is an explanatory diagram illustrating the setting of marker regions and digitalization of a relative position of the irradiation sectional region, both of which are carried out by the high sound pressure irradiation region setting process.

FIG. 11 is a flowchart showing the operation of an ultrasonic beam control process according to the embodiment.

FIG. 12 is an explanatory diagram illustrating a sound pressure distribution on sound rays of an ultrasonic beam.

FIG. 13 is an explanatory diagram depicting a beam profile at a focus depth position of the ultrasonic beam.

FIG. 14 is an explanatory diagram showing dominant regions drawn or projected on the section A.

FIG. 15 is a flowchart illustrating the operation of a high sound pressure irradiation region resetting process according to the embodiment.

FIG. 16 is an explanatory diagram showing detection of marker region positions and the calculation of a center position of the irradiation sectional region.

FIG. 17 is an explanatory diagram illustrating a method for generating an irradiation sectional region from the detected marker region positions.

FIG. 18 is an explanatory diagram showing vascular channels and dominant regions.

### DETAILED DESCRIPTION OF THE INVENTION

Embodiments of an ultrasonic imaging apparatus will be explained below with reference to the accompanying drawings. Incidentally, the invention is not limited thereby.

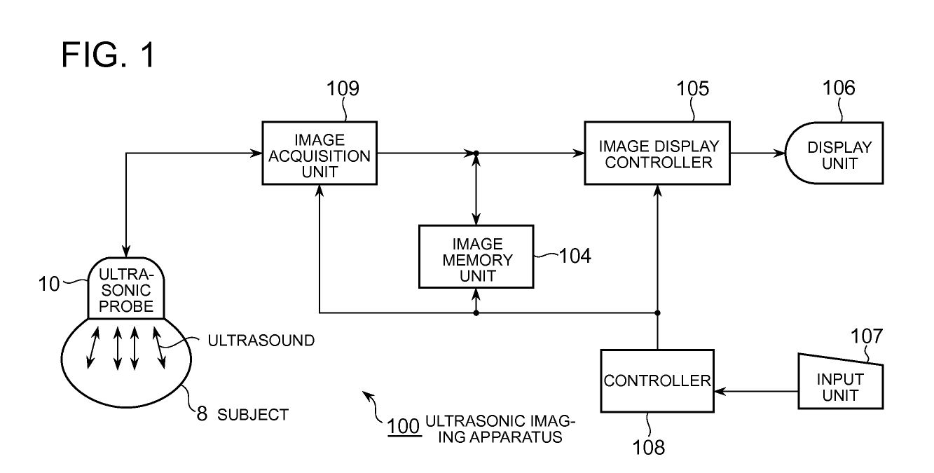

An overall construction of an ultrasonic imaging apparatus 100 according to the present embodiment will first be described. FIG. 1 is a block diagram showing the overall construction of the ultrasonic imaging apparatus 100 according to the present embodiment. The ultrasonic imaging apparatus 100 has an ultrasonic probe 10, an image acquisition unit 109, an image memory 104, an image display controller 105, a display unit 106, an input unit 107 and a controller 108.

The ultrasonic probe 10 applies ultrasound in a specific direction of an imaging section of a portion, i.e., a subject 8 for transmitting and receiving the ultrasound and receives ultrasonic echoes reflected on a case-by-case basis from inside the subject 8 as time-series sound rays. On the other hand, the ultrasonic probe 10 performs an electronic scan and a mechanical scan while the direction of irradiation of the ultrasound is being switched sequentially. As will be described in detail later, the ultrasonic probe 10 includes a piezoelectric transducer array in which piezoelectric transducers are arranged in an electronic scan direction in array form, and a mechanical scan unit for mechanically scanning the piezoelectric transducer array in the direction orthogonal to this arrangement and acquires three-dimensional tomographic image information from a three-dimensional imaging region corresponding to a first region located inside the subject 8.

The image acquisition unit 109 includes a transmission-reception part, a B mode processor, a doppler processor, etc. The transmission-reception part is connected to the ultrasonic probe 10 via a coaxial cable. The transmission-reception part generates an electric signal for driving each piezoelectric transducer of the ultrasonic probe 10 and also performs first-stage amplification of each reflected ultrasonic echo received thereat. The transmission-reception part has a drive voltage varying device and adjusts a drive voltage at the transmission of each ultrasonic echo, by extension, changes the magnitude of sound pressure held by the ultrasound lying in the subject 8.

The B mode processor performs a process for generating, in real time, a B mode image from the reflected ultrasonic echo signal amplified by the transmission-reception part. The doppler processor extracts phase change information from the reflected ultrasonic echo signal amplified by the transmission-reception part and calculates blood flow information such as an average velocity corresponding to an average frequency value of frequency shift, a power value and dispersion in real time.

The image memory 104 is of a mass storage memory which stores B mode image information, doppler image information and three-dimensional image information or the like acquired by the image acquisition unit 109 therein. The three-dimensional image information is of image information obtained by combining B mode image information different in imaging position and doppler image information. The image memory 104 is configured using, for example, a hard disk or the like.

The image display controller 105 performs conversion of display frame rates of the B mode image information generated by the B mode processor and the blood flow image information or the like generated by the doppler processor, and control on the shape and position of each image display.

The display unit 106 includes a CRT (Cathode Ray Tube) or an LCD (Liquid Crystal Display) or the like and displays a B mode image or a doppler image or the like.

The input unit 107 consists of a keyboard, a mouse and the like and is inputted with operation information from an operator. The input unit 107 performs, for example, an operation input for selecting a display based on a B mode or a display based on a doppler process, the setting of the position of a region or region for performing processing on displayed image information by a cursor or the like, designation of a region of interest, an operation input for inputting the setting of a B mode process and a doppler process, etc.

The input unit 107 inputs information such as a scan mode, a mechanical scan speed or rate, the maximum swing angle and a scan start or the like at the time that the piezoelectric transducer array of the ultrasonic probe 10 is mechanically scanned, to the controller 108.

The controller 108 controls the operations of the respective parts of the ultrasonic imaging apparatus 100 including the ultrasonic probe 10, based on operation information inputted from the input unit 107, and programs and data stored in advance. For example, the controller 108 controls the position of the piezoelectric transducer array lying inside the ultrasonic probe 10, based on the scan mode, the mechanical scan rate, the maximum swing angle and the scan start or the like of the ultrasonic probe 10, which have been inputted from the input unit 107.

The controller 108 controls a destruction ultrasonic beam corresponding to a second ultrasonic beam for destructing a contrast agent administered to the subject, using the three-dimensional tomographic image information stored in the image memory 104 updated in real time. Incidentally, this control will be described in detail later.

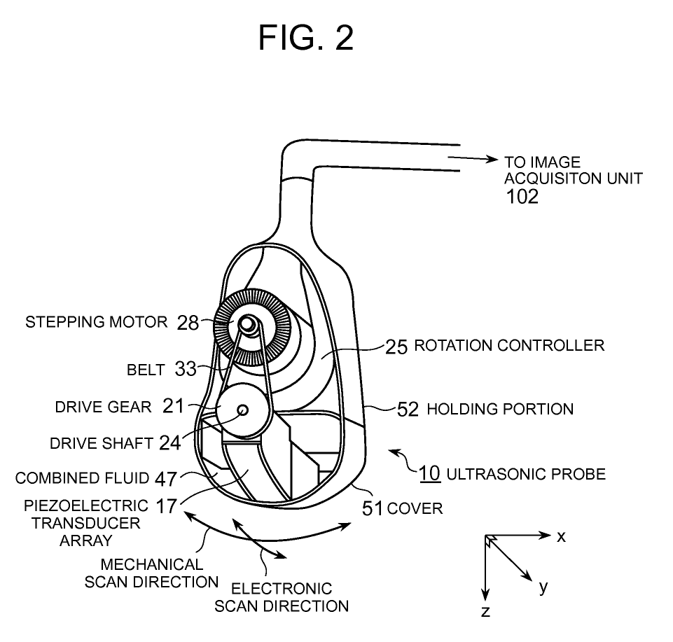

FIG. 2 is a sectional view showing an internal structure of the ultrasonic probe 10. The ultrasonic probe 10 includes a cover 51, a grasping or holding portion 52, a piezoelectric transducer array 17 and combined fluid 47, and a drive gear 21, a drive shaft 24, a stepping motor 28, a belt 33 and a rotation controller 25 which configure the mechanical scan unit. Here, the cover 51 and the holding portion 52 form a container which internally includes the piezoelectric transducer array 17 and the combined fluid 47, and the drive gear 21, the stepping motor 28, the belt 33 and the rotation controller 25 that form the mechanical scan unit. Incidentally, xyz coordinate axes shown in the figures are coordinate axes common to all drawings in which the corresponding coordinate axes exist, and indicate a position relationship between the drawings. Here, the x axis faces in an electronic scan direction, the y axis faces in a mechanical scan direction, and the z axis faces in the longitudinal direction of the holding portion 52.

The cover 51 is made up of a semitransparent film and has an arc-like shape that extends along the track of the piezoelectric transducer array 17 mechanically scanned in an arc-shaped manner. The cover 51 is formed as a material having acoustic impedance for allowing an ultrasound generated at the piezoelectric transducer array 17 and each reflected ultrasonic echo from the subject 8 to pass therethrough with a low loss.

The holding portion 52 includes moldable or shapeable plastic or the like. The holding portion 52 is brought into such a shape that the operator is capable of holding the ultrasonic probe 10 easily and reliably.

The piezoelectric transducer array 17 is of a convex linear scan type probe. This linear scan type probe has a piezoelectric transducer array in which a plurality of piezoelectric transducers are one-dimensionally arranged in the electronic scan direction approximately orthogonal to the mechanical scan direction. The linear scan type probe performs an electronic scan along this arrangement.

The mechanical scan unit scans the piezoelectric transducer array 17 in the mechanical scan direction. The mechanical scan unit has the drive shaft 24 corresponding to a swing device that faces in the electronic scan direction. With the rotation of the drive shaft 24, the surface of the probe brought into contact with the cover 51 of the piezoelectric transducer array 17 performs, in the mechanical scan direction, a swing operation for plotting or drawing an arc-shaped trajectory. Incidentally, the combined fluid 47 is charged inside the cover 51 in which the piezoelectric transducer array 17 exists, and brings acoustic coupling between the piezoelectric transducer array 17 and the cover 51 to a loss-reduced state.

The drive shaft 24 is mechanically connected to the stepping motor 28 via the drive gear 21 and the belt 33. With the input of a control pulse from the rotation controller 25, the stepping motor 28 performs a rotation at an aimed predetermined angle with high accuracy. With its rotation, the mechanically-connected drive shaft 24 and the piezoelectric transducer array 17 coupled to the drive shaft 24 are rotated in the mechanical scan direction.

The rotation controller 25 has a pulse generation unit for generating a pulse for driving the stepping motor 28, and a pulse control unit for controlling the pulse. The rotation controller 25 controls the rotational angle of the stepping motor 28, by extension, the piezoelectric transducer array 17, based on the control information sent from the image acquisition unit 109 to cause the piezoelectric transducer array 17 to perform a swing operation with the drive shaft 24 as the center of rotation.

The rotation controller 25 sets, for example, the location of the piezoelectric transducer array 17 in the z-axis direction in which the piezoelectric transducer array 17 faces the front face of the subject, as a home position and sets it as a location where it always stays when no scan is done. The rotation controller 25 starts a scan in a predetermined mechanical scan direction from the home position, based on information about the maximum swing angle of the piezoelectric transducer array 17, which has been measured from the imaging front face and information about a scan rate of the piezoelectric transducer array 17 in the mechanical scan direction, both of which are inputted by the operator. Thereafter, the rotation controller 25 brings back the piezoelectric transducer array 17 to the home position in accordance with the instructions of a scan stop from the input unit 107 by the operator and stops the scan.

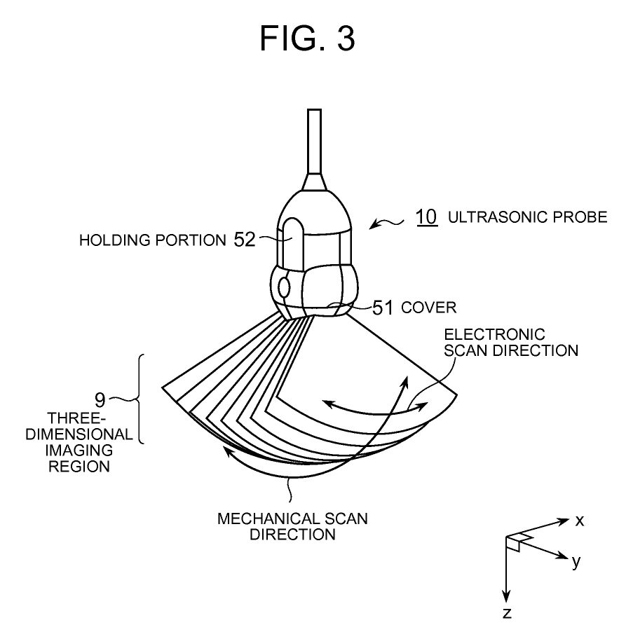

FIG. 3 is an explanatory diagram typically showing scans in an electronic scan direction and a mechanical scan direction approximately orthogonal to the electron scan direction, both of which are performed using the ultrasonic probe 10, and a three-dimensional imaging region 9 located inside the subject 8, which is acquired upon the scans. The ultrasonic probe 10 performs an electronic scan while applying a first ultrasonic beam set from the input unit 107 in the electronic scan direction of the piezoelectric transducer array 17 with the piezoelectric transducers arranged therein, thereby to acquire two-dimensional tomographic image information. Thereafter, the ultrasonic probe 10 moves the piezoelectric transducer array 17 in the mechanical scan direction orthogonal to the electronic scan direction and performs the electronic scan again thereat thereby to repeat the acquisition of tomographic image information. Thus, the three-dimensional tomographic image information in the three-dimensional imaging region 9 lying inside the subject 8 is repeatedly acquired. Incidentally, the first ultrasonic beam is defined as sound pressure that does not destroy the contrast agent lying inside the subject 8.

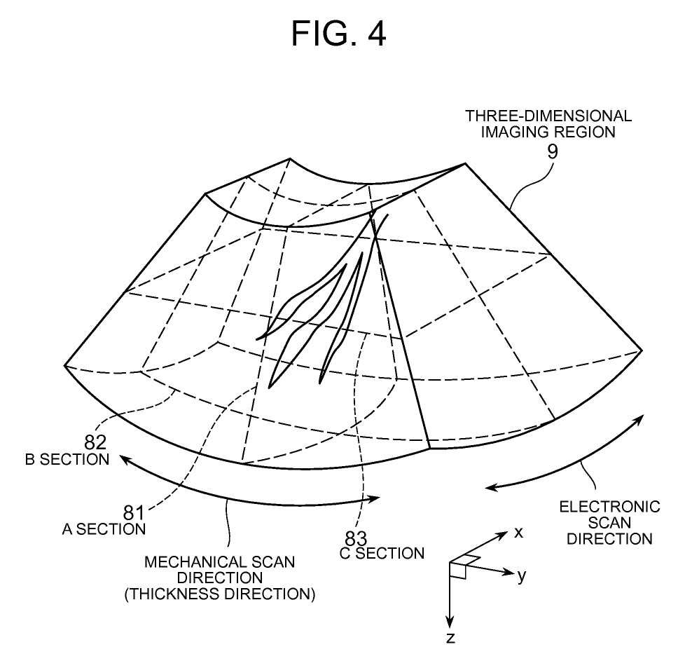

FIG. 4 is an explanatory diagram showing three-dimensional imaging region 9 corresponding to an acquired first region and a two-dimensional imaging region of the three-dimensional imaging region 9 displayed on the display unit 106. Image information about a specific two-dimensional imaging section contained in the acquired three-dimensional tomographic image information is displayed on a two-dimensional display screen of the display unit 106 in real time. For example, the display unit 106 is capable of displaying three orthogonal sections set to the three-dimensional imaging region 9 in real time.

The three orthogonal sections include an A section 81, a B section 82 and a C section 83. The A section 81 is of a section parallel to an xz-axis surface and indicates a section at the home position as viewed in the electronic scan direction. The B section 82 is of a section parallel to a yz-axis surface and indicates a section as viewed in the mechanical scan direction. The C section 83 is of a section parallel to an xy-axis surface and indicates an opposite section opposite to a contact surface at which the ultrasonic probe 10 is brought into contact with the subject 8. Incidentally, the position of the A section 81 as viewed in the mechanical scan direction, the position of the B section 82 as viewed in the electronic scan direction and the depth position of the C section 83 as viewed from the contact surface at which the ultrasonic probe 10 contacts the subject 8, can be changed by the designation given from the input unit 107. Incidentally, an arterial vessel entered or led from a portal vein and three vascular channels that branch off from the arterial vessel are illustrated in the three-dimensional imaging region 9 corresponding to the first region shown in FIG. 4. The arterial vessel and these vascular channels have three-dimensionally spread spatial configurations and do not fit into the two-dimensional imaging sections.

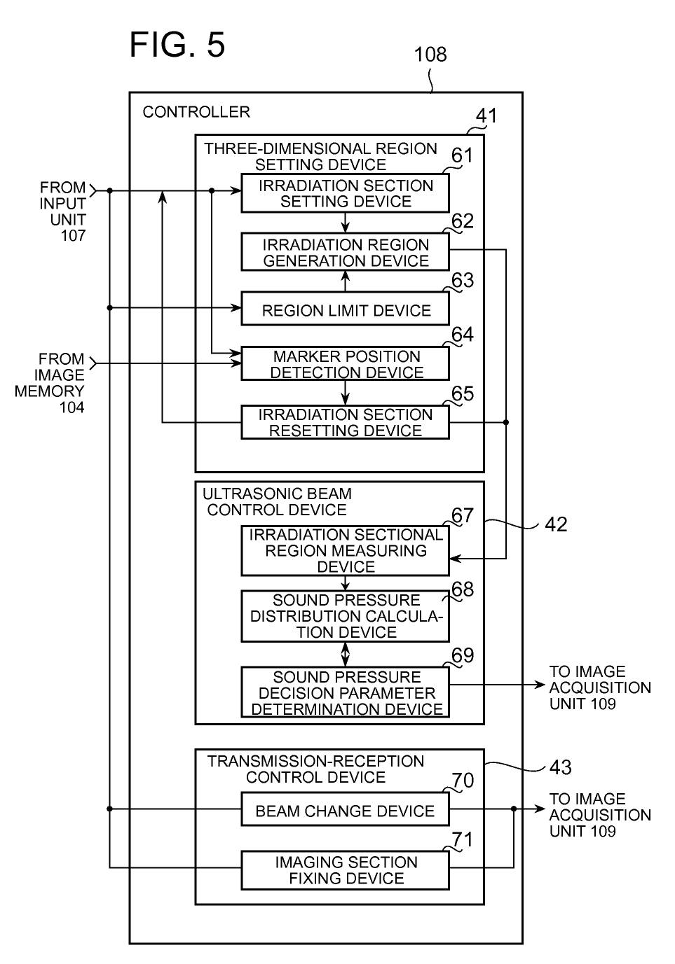

FIG. 5 is a block diagram showing a functional construction of the controller 108. The controller 108 includes a three-dimensional region setting device 41 corresponding to a region setting device, an ultrasonic beam control device 42 and a transmission-reception control device 43. The three-dimensional region setting device 41 sets a high sound pressure irradiation region corresponding to a second region to the three-dimensional imaging region 9. A destruction ultrasonic beam corresponding to a second ultrasonic beam is applied to the high sound pressure irradiation region. The destruction ultrasonic beam has destruction sound pressure for destroying the contrast agent in the high sound pressure irradiation region and has sound pressure less than the destruction sound pressure in a region other than the high sound pressure irradiation region.

The ultrasonic beam control device 42 controls a sound pressure distribution of the destruction ultrasonic beam corresponding to the second ultrasonic beam in such a manner that the sound pressure distribution has the sound pressure for destroying the contrast agent only in the high sound pressure irradiation region corresponding to the second region.

The transmission-reception control device 43 transmits a destruction ultrasonic beam to the corresponding high sound pressure irradiation region of the subject 8 in such a manner that the acquisition of three-dimensional tomographic image information having a real-time characteristic is not impaired.

Here, the three-dimensional region setting device 41 corresponding to the region setting device includes an irradiation section setting device 61, an irradiation region generation device 62, a region restricting or limit device 63, a marker position detection device 64 and an irradiation section resetting device 65. The ultrasonic beam control device 42 includes an irradiation sectional region measuring device 67, an acoustic or sound pressure distribution calculation device 68 and a sound pressure decision parameter determination device 69. The transmission-reception control device 43 includes a beam replacing or change device 70 and an imaging section fixing device 71. The detailed constructions and functions of these three-dimensional region setting device 41, an ultrasonic beam control device 42 and a transmission-reception control device 43 will be explained in detail as to the following operation of the controller 108.

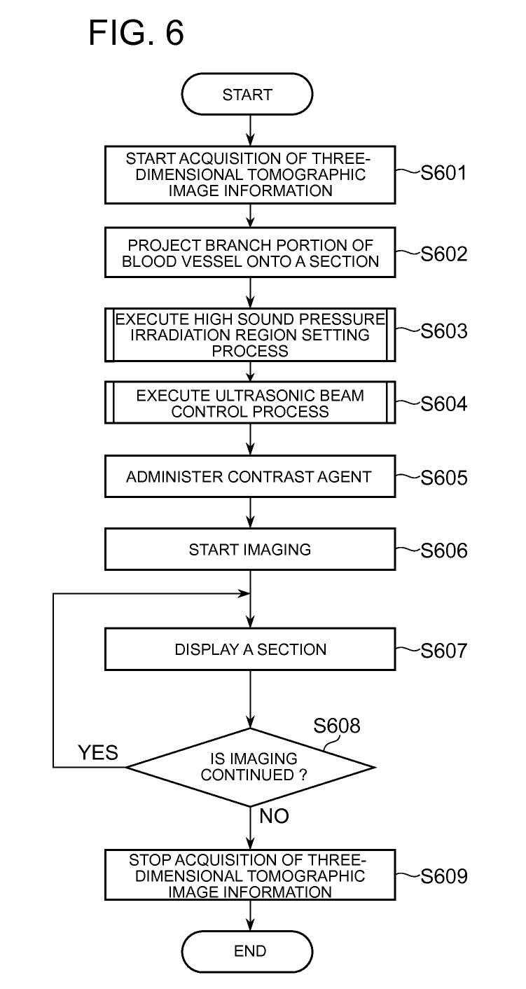

The operation of the controller 108 will next be explained using FIG. 6. FIG. 6 is a flowchart showing the operation of the controller 108. The operator first brings the ultrasonic probe 10 into intimate contact with an imaged region of the subject 8 and starts the acquisition of three-dimensional tomographic image information (Step S601). Two-dimensional tomographic image information about the A section 81, B section and C section shown in FIG. 4 are displayed on the display unit 106.

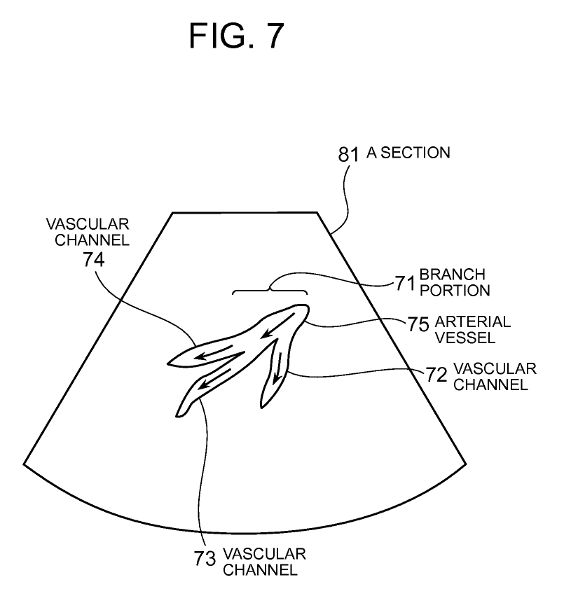

Thereafter, the operator operates or controls the ultrasonic probe 10 to draw or project, for example, a branch portion or region at which the liver of the subject 8, particularly, the arterial vessel extending from the portal vein to the liver branch into the vascular channels, on the A section 81 on which the electronic scan is done (Step S602). FIG. 7 is an explanatory diagram showing one example of the A section 81 displayed on the display unit 106. A branch portion 71 at which an arterial vessel 75 lying in the liver is branched into vascular channels 72 through 74, is illustrated in FIG. 7. The arterial vessel 75 and the vascular channels 72 through 74 respectively have structures three-dimensionally expanded even in the thickness direction orthogonal to the sheet of two-dimensional tomographic image information shown in FIG. 7. Incidentally, each of arrows shown in the arterial vessel 75 indicates the direction in which the blood flows.

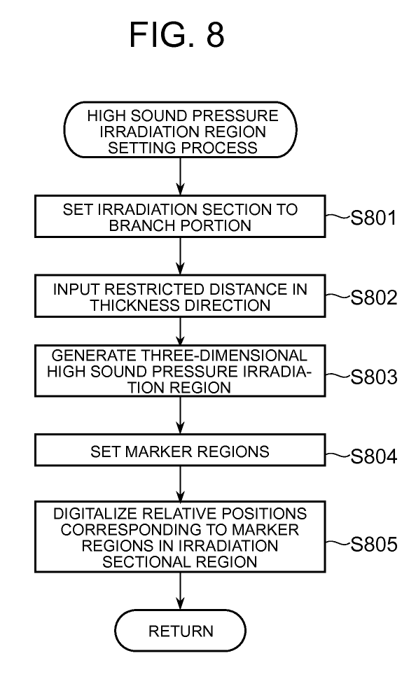

Thereafter, the operator performs a high sound pressure irradiation region setting process through the three-dimensional region setting device 41 corresponding to the region setting device (Step S603). FIG. 8 is a flowchart showing the operation of the high sound pressure region setting process, which is performed by the three-dimensional region setting device 41. The operator performs the designation of a position of such a branch portion 71 shown in FIG. 7 on its image by using a cursor or the like (Step S801). The irradiation section setting device 61 sets an irradiation sectional region to the A section 81, based on information about the designated position. The irradiation section setting device 61 sets this irradiation sectional region as an A section of a high sound pressure irradiation region corresponding to a three-dimensional second region.

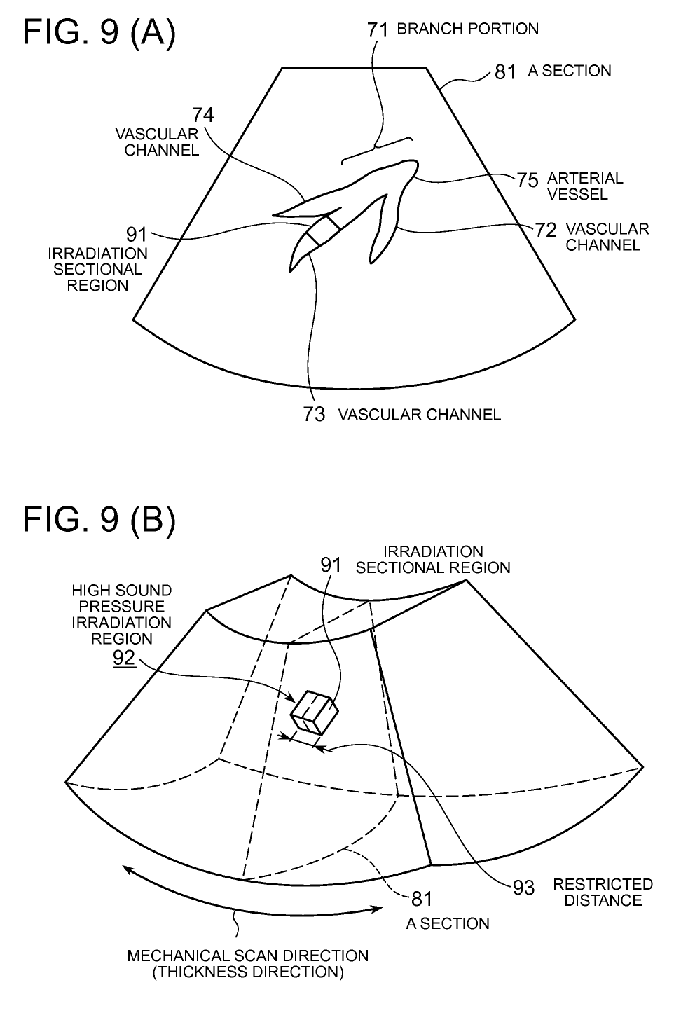

FIG. 9(A) shows one example of a case in which an irradiation sectional region 91 is set to the A section 81 shown in FIG. 7. In FIG. 9(A), the arterial vessel 75 branches off at the branch portion or region 71, and the irradiation sectional region 91 is set to an entrance portion that extends to the vascular channel 73. The irradiation sectional region 91 is set along the vessel wall so as to cover a blood flow flowing into the vascular channel 73 all.

Thereafter, the operator inputs a restricted distance corresponding to the length of a thickness-direction high sound pressure irradiation region to the region limit device 63 through a restricted distance input key of the input unit 107 (Step S802). The region limit device 63 places the thickness-direction length of the high sound pressure irradiation region within the restricted distance using the restricted distance. Incidentally, the restricted distance is set approximately to a size of such a degree that it slightly exceeds the diameter of a blood vessel contained in the vascular vessel 73 of the irradiation sectional region 91.

Thereafter, the irradiation region generation device 62 generates a high sound pressure irradiation region corresponding to a three-dimensional second region, based on information about the irradiation sectional region 91 and the restricted distance (Step S803). FIG. 9(B) is an explanatory diagram showing a high sound pressure irradiation region 92 generated in a three-dimensional imaging region 9 by the irradiation region generation device 62. The high sound pressure irradiation region 92 becomes a region or region of rectangular parallelepiped, which has a restricted distance 93 in its thickness direction with the irradiation sectional region 91 as a center position. Incidentally, since the restricted distance 93 slightly exceeds the size of the diameter of the blood vessel, the region of rectangular parallelepiped includes the vascular channel 73.

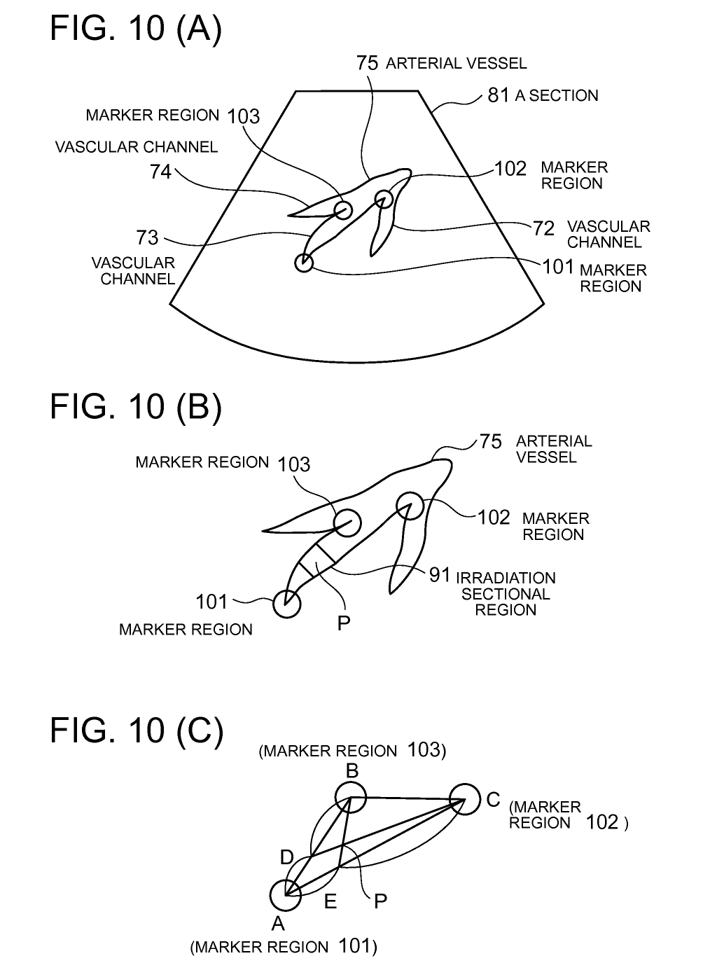

Afterwards, the operator sets marker regions 101 through 103 to the two-dimensional tomographic image information of the A section 81 (Step S804). FIG. 10(A) shows the circular marker regions 101 through 103 set onto an image of the arterial vessel when the irradiation sectional region 91 is set to the vascular channel 73 of the A section 81. Incidentally, information about the positions of the marker regions 101 through 103 are inputted to the marker position detection device 64.

As will be described later, each of the marker regions 101 through 103 designates a region for detecting a change in the position of the irradiation sectional region 91, which occurs due to body motion. Thus, the marker regions 101 through 103 are set to regions or regions suitable for detecting the movement of the vascular channel 73. In FIG. 10(A), the marker regions are set in such a manner that the center positions of the circular marker regions 101 through 103 respectively coincide with a branch point (marker region 102) between the vascular channels 72 and 73, a branch point (marker region 103) between the vascular channels 73 and 74, and a leading end or tip portion (marker region 101) of the vascular channel 73 at the A section 81. These points move in cooperation with the change in the position of the irradiation sectional region 91 and include tip-sharpened points at an image. It is easy to detect the change in position.

Thereafter, the marker position detection device 64 digitalizes the relative positions relative to the marker regions 101 through 103 of the irradiation sectional region 91 (Step S805). The marker position detection device 64 determines the center positions of the respective marker regions, based on information of the irradiation sectional region 91 and information on the positions of the marker regions 101 through 103 from the irradiation section setting device 61. FIG. 10(B) shows only the arterial vessel 75 and the marker regions 101 through 103 extracted from the image of the A section 81 shown in FIG. 10(A). The set irradiation sectional region 91 is illustrated in the image.

FIG. 10(C) shows only the marker regions 101 through 103 and the center position P of the irradiation sectional region 91 extracted from the drawing illustrative of the arterial vessel 75, marker regions 101 through 103 and irradiation sectional region 91 shown in FIG. 10(B). The center positions of the marker regions 101 through 103 are expressed in A, B and C respectively. At this time, let's assume that a point where a linear curve that passes through points C and P and a line segment AB intersect, is defined as D, and a point where a linear curve that passes through points B and P and a line segment AC intersect is defined as E. Here, the marker position detection device 64 determines a ratio between AD:DB and AE:EC. This ratio is defined as the center position P of the irradiation sectional region 91 relative to the marker regions 101 through 103 typified by the ABC points. The present high sound pressure irradiation region setting process is terminated.

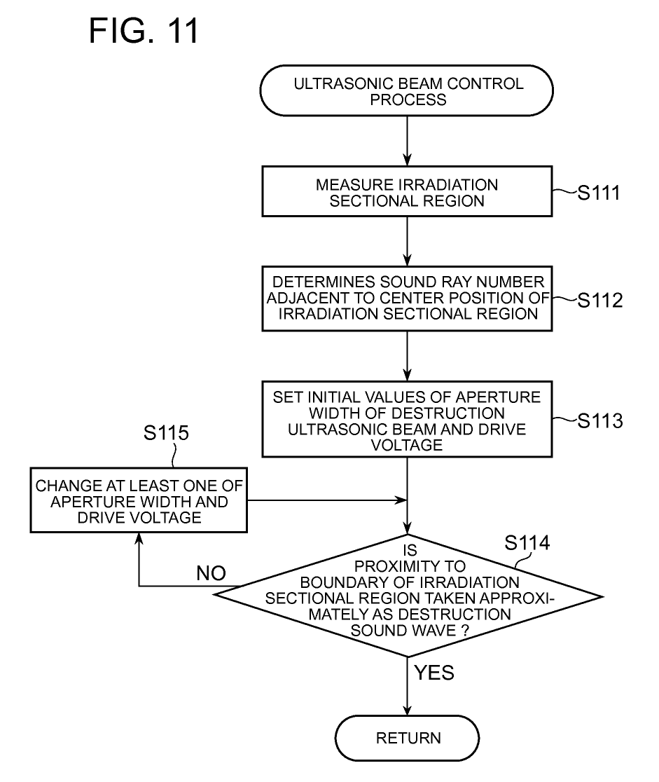

Referring back to FIG. 6 subsequently, the ultrasonic beam control device 42 performs an ultrasonic beam control process (Step S604). FIG. 11 is a flowchart showing the operation of the ultrasonic beam control process. The irradiation sectional region measuring device 67 performs the measurement of the irradiation sectional region 91 (Step S111). Upon this measurement, the depth position in the depth direction, of the center position P that the irradiation sectional region 91 has, and the region or region width L in the electronic scan direction, of the irradiation sectional region 91 are determined. The irradiation sectional region measuring device 67 determines a sound ray number of each sound ray, close to the center position P (Step S112). The sound ray indicates a line indicative of a path for the intrusion of an ultrasonic beam transmitted in the depth direction of the subject 8 from the surface of the piezoelectric transducer array 17. The sound ray number indicates the position of transmission of the ultrasonic beam in the direction of arrangement of the piezoelectric transducer array 17.

Thereafter, the ultrasonic beam control device 42 sets initial values of an opening or aperture width and a drive voltage corresponding to sound pressure decision parameters where the destruction ultrasonic beam is applied to the subject 8, to the sound pressure distribution calculation device 68 (Step S113). The initial value of the aperture width is defined as the maximum aperture width set every sound ray number. The maximum aperture width is set to an aperture or opening width equal to about twice the aperture width taken when imaging is performed, at the center in the direction of an arrangement of the piezoelectric transducer array 17. The initial value of the drive voltage is set to a voltage equal to about half the voltage at the time that imaging is performed. Here, the ultrasonic beam control device 42 calculates an irradiation sectional region 91 and a sound pressure distribution in the neighborhood thereof through the sound pressure distribution calculation device 68 where it has the values of the set sound pressure decision parameters. The sound pressure distribution calculation device 68 will be explained below.

The sound pressure distribution calculation device 68 determines sound pressure in proximity to the boundary of the irradiation sectional region 91 using information about the aperture width and drive voltage corresponding to the set sound pressure decision parameters. The sound pressure distribution calculation device 68 determines each sound pressure amplitude Pm in water or moisture from scan parameter values and an experimentally measured sound pressure distribution in water. A distribution of a sound pressure amplitude Pm in water on the basis of predetermined scan parameter values is experimentally determined in advance by the movement or the like of a hydrophone installed in water, for example. This is set in advance to the sound pressure distribution calculation device 68 as non-volatile information inputted by hand from the input unit 107 or written in an ROM or the like.

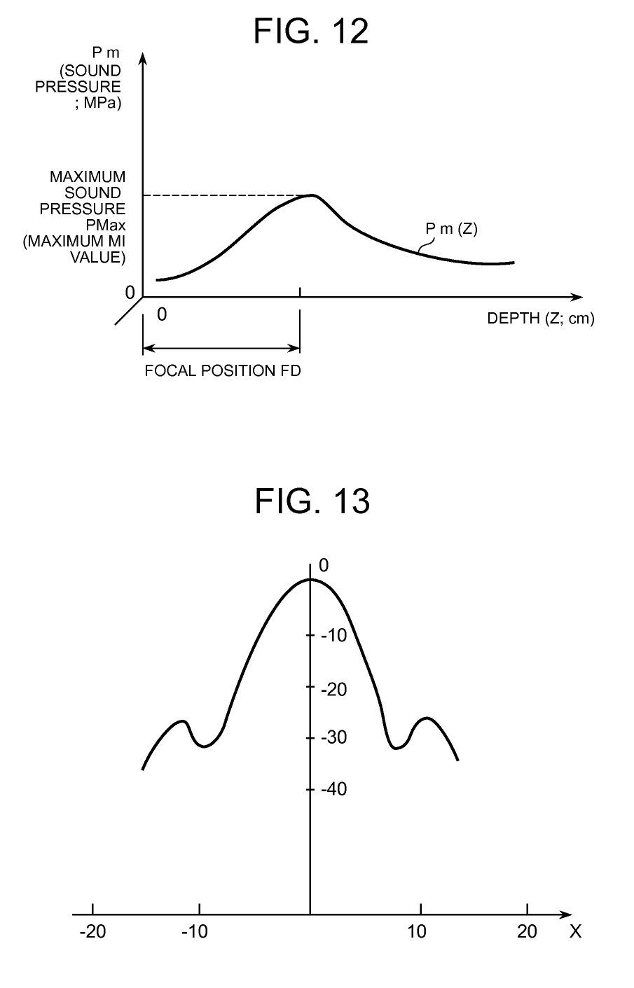

FIG. 12 is an explanatory diagram showing one example of an acquired sound pressure distribution function. In FIG. 12, the direction of a depth extending inside the subject 8 as viewed from the contact surface between the ultrasonic probe 10 and the subject 8 is defined as the horizontal axis (z axis), and the sound pressure amplitude Pm indicated by an irradiated ultrasound is defined as the vertical axis. Here, the z axis indicative of the horizontal axis is defined with the surface of the ultrasonic probe 10 brought into contact with the subject 8 being assumed to be the origin point. A sound pressure distribution in the depth direction shows the maximum sound pressure PMax at a position close to a focal depth FD and thereafter decreases in sound pressure.

Such a sound pressure distribution function Pm(Z) shown in FIG. 12 changes according to a change in each scan parameter value. As the scan parameter values that change the sound pressure distribution, may be mentioned, for example, probe information Ty including the resonant frequency of the ultrasonic probe 10 and the like, a focal depth FD of an electronic focus done in a scan direction, an opening or aperture width AW indicative of the number of piezoelectric transducers simultaneously driven in the scan direction, apodization information AP and a drive voltage MV for driving each piezoelectric transducer. The value of the experimentally determined sound pressure distribution function Pm(Z) is corrected to a suitable value by these scan parameter values. Assuming that this correction function is f, the value PM of the sound pressure distribution function Pm(Z) is corrected as follows:

PM=f(Pm,MV,Ty,FD,AW,AP, . . . )

Incidentally, the correction function f has a complex functional form. A corrected PM on the left side is determined from an operation or computational part on the right side.

A beam profile indicative of a sound pressure distribution spread in an x-axis direction orthogonal to the depth direction at the position of the focal depth FD is determined by calculation with respect to the depth-direction sound pressure distribution function Pm(Z). Relative sound pressure PR in the x-axis direction with respect to the sound pressure PM at the center position becomes a function of the x-axis direction position X, aperture width AW, focal depth FD and apodization information AP. Assuming that this function is g, the relative sound pressure can be expressed as follows:

PR=g(X,FD,AW,AP)

PR is inversely proportional approximately to the aperture width AW.

FIG. 13 is an explanatory diagram showing one example of the beam profile. The horizontal axis indicates the position from each sound ray in an x-axis direction indicating a scan direction. The vertical axis indicates relative sound pressure expressed in decibel. When the center position of the ultrasonic beam x=0, the sound pressure distribution indicates a peak and the sound pressure is reduced with the movement of the ultrasonic beam to both sides in the x-axis direction. Accordingly, the sound pressure P(X) at the position X in the x-axis direction at the focal depth FD is expressed as follows:

P(X)=PM×PR

Here, the sound pressure in proximity to the boundary of the irradiation sectional region 91 is made approximate to sound pressure at a position separated by a half L/2 of the region or region width L from the center position. Thus, the sound pressure in proximity to the boundary of the irradiation sectional region 91 set to the focal depth FD can be determined by P (L/2) with respect to the set aperture width AW and drive voltage MV.

Referring back to FIG. 11 subsequently, the sound pressure decision parameter determination device 69 determines whether the sound pressure P (L/2) in proximity to the boundary of the irradiation sectional region 91 takes or assumes destruction sound pressure PD of a contrast agent (Step S114). When P (L/2)≅PD is denied (NO at Step S114), the sound pressure decision parameter determination device 69 changes at least one of the aperture width AW and the drive voltage MV (Step S115). If P (L/2)<PD, for example, then the drive voltage MV is raised or the aperture width AW is increased. If P (L/2)>PD, then the drive voltage MV is lowered or the aperture width AW is decreased. Then, the ultrasonic beam control process proceeds to Step S114, where the sound pressure in proximity to the boundary of the irradiation sectional region 91 and the destruction sound pressure are compared again.

When P (L/2) PD (YES at Step S114), the aperture width AW and the drive voltage MV set the sound pressure to the destruction sound pressure or more inside the irradiation sectional region 91 and set the sound pressure to the destruction sound pressure or less outside the irradiation sectional region 91. Therefore, the sound pressure decision parameter determination device 69 sets the values as parameter values at the transmission of the destruction ultrasonic beam corresponding to the second ultrasonic beam, and the present process is terminated.

Referring back to FIG. 6 subsequently, the operator administers the contrast agent to the subject 8 (Step S605), and the beam change device 70 of the transmission-reception control device 43 is selected to start the imaging of the three-dimensional imaging region 9 corresponding to the first region (Step S606). When the acquisition of three-dimensional tomographic image information using the ultrasonic probe 10 is performed, the beam change device 70 transmits the destruction ultrasonic beam corresponding to the second ultrasonic beam at a sound ray number position where the irradiation sectional region 91 exists, and does not perform transmission/reception for acquiring tomographic image information. Thus, the beam change device 70 performs the destruction of the contrast agent while performing the acquisition of the three-dimensional tomographic image information. Therefore, the display frame rate of the A section 81 displayed is not affected by the destruction of the contrast agent. Incidentally, as the contrast agent to be administered, one (Sonazoid®, which is a registered trademark of Amersham Health AS Corporation, Oslo, Norway) is used in which if a contrast effect repeatedly appears with the repetitive irradiation of the ultrasonic beam if the sound pressure is less than the destruction sound pressure of the contrast agent.

Thereafter, the controller 108 displays the A section 81 on the display unit 106 (Step S607) and observes a change in the second-dimensional tomographic image information shown in FIG. 9(A). Here, the destruction ultrasonic beam is sequentially applied to the irradiation sectional region 91 corresponding to the entrance of a blood flow to the vascular channel 73. Thus, the contrast agent is prevented from flowing into the tip of the vascular channel 73 at which the marker region 101 exists. On the other hand, since the contrast agent that flows into the vascular channels 72 and 74 is not destroyed, the contrast agent is absorbed from the peripheral blood vessels of the vascular channels 72 and 74 to tissues that form the dominant regions or regions of the respective vascular channels.

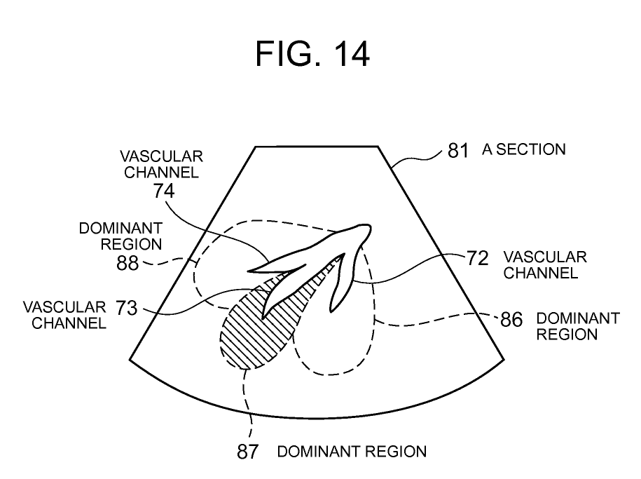

FIG. 14 is an explanatory diagram typically showing this state of the A section 81. Dominant regions or regions 86 through 88 of the vascular channels 72 through 74 with respect to the respective vascular channels are illustrated in FIG. 14. Since the contract agent flows into the dominant regions 86 and 88 and is absorbed into cells, the dominant regions 86 and 88 form high brightness regions or regions respectively. On the other hand, since the contrast agent does not flow into the dominant region 87, the dominant region forms a low brightness region or region. It is thus possible to confirm the dominant region 87 of the vascular channel 73 experimentally.

Thereafter, the operator determines whether the imaging should be continued while the dominant region 87 of the vascular channel 73 is being observed (Step S608). When it is desired to further store the contrast agent in the dominant regions 86 and 88 of the vascular channels 72 and 74 and recognize the dominant region 87 clearer, the operator continues imaging and continues the display of the A section 81 (YES at Step S608). When the observation of the dominant region 87 is ended, the operator terminates the imaging (NO at Step S608) and stops the acquisition of three-dimensional tomographic image information (Step S609). Then, the operator terminates the present process.

In the preset embodiment as described above, the irradiation sectional region 91 is provided at the entrance portion of the vascular channel 73, which branches off from the arterial vessel 75, and the destruction ultrasonic beam of high sound pressure for destroying the contrast agent is applied to the contrast agent that passes through the irradiation sectional region 91. Therefore, the contrast agent is absorbed into the dominant regions 86 and 88 of the vascular channels 72 and 74, whereas the contrast agent is prevented from being absorbed into the dominant region 87 of the vascular channel 73. Further, only the dominant region 87 is projected onto the A section 81 as the low brightness region, and the actual dominant region 87 of vascular channel 73 can be visually recognized.

In the present embodiment, the transmission-reception control device 43 has performed the transmission of the destruction ultrasonic beam corresponding to the second ultrasonic beam through the beam change device 70 in the course of the acquisition of the three-dimensional tomographic image information. It is however also possible to provide the imaging section fixing device 71 additionally, stop the scan in the mechanical scan direction by the imaging section fixing device 71 and repeat an electronic scan of the same imaging section thereby to acquire an image of the A section 81 at a high frame rate. Incidentally, since the repetition cycle or period in which the destruction ultrasonic beam is applied to the high sound pressure irradiation region corresponding to the second region becomes earlier in this method, the high sound pressure irradiation region can be used when the blood flows at a high rate.

In the present embodiment, the irradiation region generation device 62 has used, as the high sound pressure irradiation region, the region of rectangular parallelepiped in which the irradiation sectional region is expanded in the thickness direction corresponding to the mechanical scan direction. However, it is also possible to make coincidence with the average pixel value of the irradiation sectional region 91 within the threshold value and to calculate an adjacent thickness-direction three-dimensional same pixel value region and set a region in which the width of the three-dimensional same pixel value region in the thickness direction with the A section 81 as the center is within the restricted distance, as a high sound pressure irradiation region.

In the present embodiment, the thickness direction corresponding to the mechanical scan direction of the high sound pressure irradiation region is set as the restricted distance at Step S803 of the high sound pressure irradiation region setting process by the operator. When the restricted distance is longer than the thickness of the destruction sound pressure beam in the thickness direction, each sound ray number in the electronic scan direction remains identical. In this state, the destruction ultrasonic beam corresponding to the second ultrasonic beam is applied to the subject 8 through a plurality of frames different in thickness-direction position, and the contrast agent is destroyed over the full range of the high sound pressure irradiation region.

In the present embodiment, the three-dimensional region setting device 41 corresponding to the region setting device starts imaging at Step S606 of the main routine, and thereafter the irradiation section region 91 remains fixed. It is however also possible to take into consideration the change in the position of the vascular channel 73 due to the body motion of the subject 8 and provide the irradiation section resetting device 65 for automatically correcting the position of the irradiation sectional region 91.

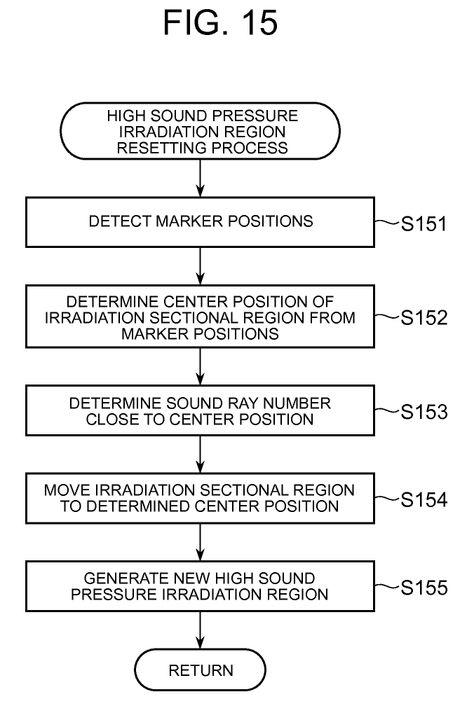

FIG. 15 is a flowchart showing the operation of a high sound pressure irradiation region resetting process executed at the three-dimensional region setting device 41. Incidentally, the high sound pressure irradiation region resetting process is performed in the course of the acquisition of the three-dimensional tomographic image information, i.e., between Step S607 and Step S608 of the main routine. The marker position detection device 64 detects the positions of the marker regions 101 through 103 set by the irradiation section setting device 61 using the acquired two-dimensional tomographic image information of the A section 81 (Step S151). Upon this detection, the regions or regions designated by the marker regions 101 through 103, and such a circular retrieval region as to include the regions and the moving ranges of the marker regions 101 through 103 sufficiently are set. For example, a circular retrieval region or region having a radius equivalent to the length equal to about half the distance between the marker regions 101 and 102 with the maker region 101 at its setting as the center is set to the maker region 101.

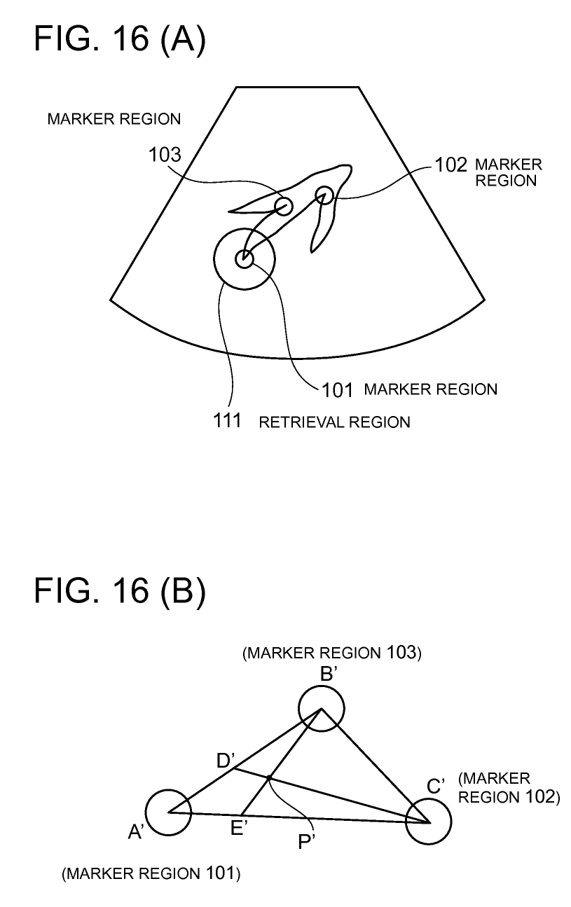

FIG. 16(A) is a diagram showing one example of a retrieval region 111 for the marker region 101. The retrieval region 111 has a radius having a length equal approximately to half the distance between the marker regions 101 and 103 with the position of the marker region 101 at its setting as the center. The position of the marker region 101 is moved by body motion. The marker position detection device 64 brings the marker region 101 and the retrieval region 111 into binary form and moves the marker region 101 within the retrieval region 111 to perform pattern matching on a difference or the like, thereby setting the position best in matching as the new position of marker region 101. Processing similar to the above is performed even on the marker regions 102 and 103 to determine new marker region positions.

Thereafter, the irradiation section resetting device 65 determines a new center position P′ of the irradiation sectional region 91, based on the new position information about the marker regions 101 through 103 (Step S152). FIG. 16(B) is an explanatory diagram showing a method for determining the new center position P′ of the irradiation sectional region 91 by the irradiation section resetting device 65. Assuming that the new positions of the marker regions 101 through 103 are A′, B′ and C′, the irradiation section resetting device 65 determines D′ E′ having a ratio equal to the ratio between AD:DB and AE:EC, which has been determined using the marker position detection device 64 at Step S805 shown in FIG. 8, with respect to A′, B′ and C′. The irradiation section resetting device 65 sets a point where C′D′ and B′E′ intersect, as a new center position P′.

Thereafter, the irradiation section resetting device 65 determines a sound ray number of each sound ray close to the center position P′ (Step S153) and moves the center position of the irradiation sectional region 91 to P′ (Step S154). Then, the irradiation region generation device 62 generates a high sound pressure irradiation region 92 corresponding to a new second region or region using the moved irradiation sectional region 91 (Step S155), and the present process is terminated.

In the present embodiment, the destruction ultrasonic beam corresponding to the second ultrasonic beam remains fixed after the imaging has been started at Step S606 of the main routine. It is however also possible to take into consideration a change in the size of the vascular channel 73 due to the movement of the irradiation sectional region 91 and the body motion of the subject 8 and control the destruction ultrasonic beam automatically. This control is done by performing the control exactly similar to the ultrasonic beam control process shown in FIG. 11, based on the movement of the irradiation sectional region 91 and the change in the size thereof.

Incidentally, the irradiation section resetting device 65 can bring the size of the irradiation sectional region 91 to one suitable for the size of the vascular channel 73, based on the changes in the positions of the marker regions 101 through 103. Upon execution of the high sound pressure irradiation region setting process, the operator designates a line segment extending along a vessel wall connecting between the marker regions, e.g., a line segment AC connecting between the marker regions 101 and 102 in the example shown in FIG. 10 simultaneously with the setting of the maker regions 101 through 103. Considering that the line segment AC maintains a state of extending approximately along the vessel wall, the distance between the new center position P′ and line segment AC referred to above becomes a length equal to half of a vessel diameter subsequently.

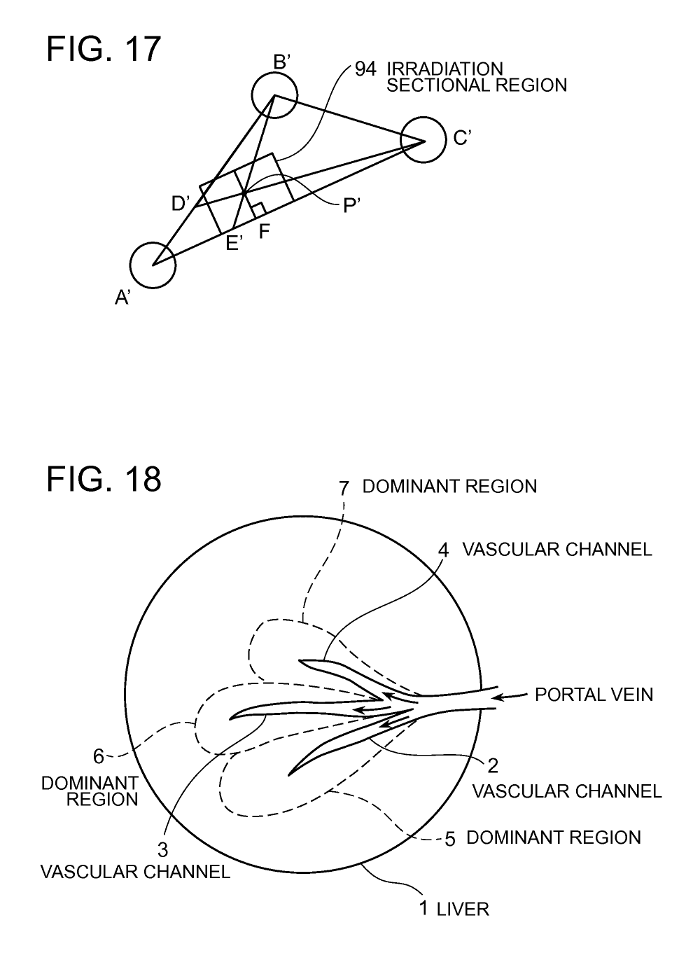

FIG. 17 is an explanatory diagram showing a method for determining each new irradiation sectional region 94 different in size from center positions A′, B′ and C′ of newly detected marker regions 101 through 103 by the irradiation section resetting device 65. D′ and E′ at which A′ D′:D′ B′ and A′E′:E′ C′ reach a predetermined ratio are determined using the center positions A′, B′ and C′ of the marker regions detected by the marker position detection device 64. A new center position P′ is determined from a point where a line segment of C′ D′ and a line segment of B′ E′ intersect.

Considering that a line segment of A′ C′ remains unchanged in a state of extending along the vessel wall here, the distance P′ F between the center position P′ and the line segment A′ C′ becomes approximately half of the vessel diameter. Accordingly, the irradiation section resetting device 65 sets a rectangular region or region that passes through a point symmetric with respect to a point F about the center position P′, as a new irradiation sectional region 94. Thus, the irradiation section resetting device 65 can set an irradiation sectional region or region having a size that approximately covers the vascular channel 73 changed in size.

Although the ultrasonic probe for mechanically scanning the piezoelectric transducer array 17 has been used in the present embodiment, an ultrasonic probe in which piezoelectric transducers are two-dimensionally arranged at the surface brought into contact with the subject 8, may be used. This ultrasonic probe is capable of acquiring all three-dimensional tomographic image information through an electronic scan at a high rate.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus having comprising:
an ultrasonic probe configured to apply a first ultrasonic beam to a first region of a three-dimensional region of a subject with a contrast agent administered thereto and to acquire three-dimensional tomographic image information for the first region;
a region setting device configured to set a second region for applying a second ultrasonic beam whose sound pressure is higher than that of the first ultrasonic beam for not destroying the contrast agent, in the three-dimensional region; and
a controller configured to enable the second ultrasonic beam to exceed sound pressure for destroying the contrast agent only in the second region and to control the irradiation of the second ultrasonic beam by said ultrasonic probe in the course of acquisition of the three-dimensional tomographic image information in the first region by the first ultrasonic beam.

2. The ultrasonic imaging apparatus according to claim 1, wherein the second region is a blood vessel that branches off within a liver of the subject.

3. The ultrasonic imaging apparatus according to claim 1, wherein said region setting device is configured to set the second region based on two-dimensional tomographic image information constituting the three-dimensional tomographic image information, said ultrasonic imaging apparatus further comprising:
a display unit configured to display an image based on the two-dimensional tomographic image information; and
an irradiation section setting device configured to set an irradiation sectional region for the second region to the image.

4. The ultrasonic imaging apparatus according to claim 3, wherein said irradiation section setting device is configured to set marker regions for detecting the motion of the irradiation sectional region to the image.

5. The ultrasonic imaging apparatus according to claim 4, wherein said region setting device comprises a marker region position detection device configured to detect a location where each of the marker regions is positioned.

6. The ultrasonic imaging apparatus according to claim 5, wherein said region setting device comprises an irradiation section resetting device configured to reset the position of the irradiation sectional region, based on information about the positions of the marker regions detected by said marker region position detection device.

7. The ultrasonic imaging apparatus according to claim 3, wherein said region setting device comprises an irradiation region generation device configured to expand the irradiation sectional region in a thickness direction orthogonal to the image and to thereby generate the second region.

8. The ultrasonic imaging apparatus according to claim 7, wherein said irradiation region generation device comprises a region limit device configured to limit a length of the second region in the thickness direction to within a predetermined restricted distance.

9. The ultrasonic imaging apparatus according to claim 8, further comprising a restricted distance input key configured to input the restricted distance.

10. The ultrasonic imaging apparatus according to claim 3, wherein said controller comprises an irradiation sectional region measuring device configured to measure a size of the irradiation sectional region in an electronic scan direction orthogonal to the direction of a depth that the image based on the two-dimensional tomographic image information has.

11. The ultrasonic imaging apparatus according to claim 3, wherein said controller comprises a sound pressure distribution calculation device configured to calculate a sound pressure distribution of the second ultrasonic beam generated by a sound ray closest to a center position of the irradiation sectional region.

12. The ultrasonic imaging apparatus according to claim 11, wherein said sound pressure distribution calculation device is configured to set a depth that the center position of the irradiation sectional region to a focal depth on which the second ultrasonic beam is focused.

13. The ultrasonic imaging apparatus according to claim 3, wherein said controller comprises a sound pressure decision parameter determination device configured to change sound pressure decision parameters used upon calculation of a sound pressure distribution.

14. The ultrasonic imaging apparatus according to claim 13, wherein the sound pressure decision parameters include an aperture width and a drive voltage used when the second ultrasonic beam is transmitted.

15. The ultrasonic imaging apparatus according to claim 14, wherein the aperture width is set to an aperture width wider than the aperture width at the time that the first transmission is done.

16. The ultrasonic imaging apparatus according to claim 3, wherein said controller comprises a beam change device configured to stop the first ultrasonic beam which acquires the two-dimensional tomographic image information at a position of the sound ray closest to the center position of the irradiation sectional region and to generate the second ultrasonic beam in place of the first ultrasonic beam, upon acquisition of the three-dimensional tomographic image information.

17. The ultrasonic imaging apparatus according to claim 1, wherein said ultrasonic probe comprises a piezoelectric transducer array in which piezoelectric transducers are arranged on a one-dimensional basis, and a mechanical scan unit configured to mechanically move said piezoelectric transducer array in a direction approximately orthogonal to a direction of the arrangement thereof.

18. The ultrasonic imaging apparatus according to claim 17, wherein upon acquisition of the two-dimensional tomographic image information by an electronic scan done in the direction of the arrangement of said piezoelectric transducer array, said controller is configured to stop the mechanical scan and to repeatedly perform only the electronic scan.

19. The ultrasonic imaging apparatus according to claim 1, wherein comprises a two-dimensional piezoelectric transducer array in which piezoelectric transducers are two-dimensionally arranged at a surface brought into contact with the subject.

20. An ultrasonic imaging method using an ultrasonic probe, said method comprising:
applying a first ultrasonic beam to a first region of three-dimensional region of a subject with a contrast agent administered thereto:
acquiring three-dimensional tomographic image information for the first region;
setting a second region for applying a second ultrasonic beam whose sound pressure is higher than that of the first ultrasonic beam for not destroying the contrast agent, in the three-dimensional region;
applying the second ultrasonic beam to exceed sound pressure for destroying the contrast agent only in the second region; and
performing the irradiation of the second ultrasonic beam by the ultrasonic probe in the course of acquisition of the three-dimensional tomographic image information in the first region by the first ultrasonic beam.
